(19) United States
(12) Patent Application Publication
(10) Pub. No.: US 2018/0060519 A1
(43) Pub. Date: Mar. 1, 2018

(54) SYSTEM AND DEVICE FOR REMOTE MEDICAL SUPPORT

(71) Applicant: Beyond Reps, Inc., Phoenix, AZ (US)

(72) Inventor: Andrew Nash, Scottsdale, AZ (US)

(21) Appl. No.: 15/680,647

(22) Filed: Aug. 18, 2017

Related U.S. Application Data
(60) Provisional application No. 62/377,037, filed on Aug. 19, 2016.

(51) Int. Cl. G06F 19/00; G06F 21/62; H04L 29/06; A61N 1/372

(52) U.S. Cl. CPC G06F 19/3418; G06F 21/6245; H04N 7/141; A61N 1/37282; A61N 1/37247; H04L 63/0464

(57) ABSTRACT

A device remotely supporting an implantable cardiac device is disclosed. The device includes at least one video input configured to receive at least a video signal from a programmer for an implantable cardiac device, at least one audio input, a network interface, a display, and a user input. The device may also include one or more hardware processors configured with instructions to transmit one or more video signals received at the at least one video input over a network to a remote location via the network interface, transmit one or more audio signals received at the at least one audio input over the network to the remote location via the network interface, receive patient data from a database on the network via the network interface, and display the patient data on the display.

### INCORPORATION BY REFERENCE TO ANY PRIORITY APPLICATIONS

This application claims the benefit of U.S. Provisional Application No. 62/377,037, filed Aug. 19, 2016, and which is incorporated herein by reference. Any and all applications for which a foreign or domestic priority claim is identified in the Application Data Sheet as filed with the present application are hereby incorporated by reference under 37 CFR 1.57.

### BACKGROUND

### Field

The present disclosure relates generally to devices, systems, and methods for providing remote medical support. In some embodiments, this disclosure provides devices, systems, and methods that can be used to provide remote support for implantable cardiac devices, such as pacemakers or implantable defibrillators, among others.

### Description

Healthcare providers commonly surgically install implantable cardiac devices in patients suffering from a variety of types of heart problems or arrhythmias. For example, an implantable artificial pacemaker can be installed which uses electrical impulses to regulate the beating of the heart. As another example, an implantable cardioverter-defibrillator (ICD) can be installed to monitor the rhythm of the heart, detect arrhythmias, and provide electrical impulses to help ensure a regular heartbeat. Such devices are referred to throughout this disclosure as “implantable cardiac devices,” and this term is used broadly to encompass all types of medical devices that may be surgically implanted in a patient for all types of cardiac treatment.

Implantable cardiac devices are available from a number of medical device manufacturers. In general, each medical device manufacturer employs a team of device representatives that provide in-person support for the implantable cardiac devices. The device representatives are available in person to program the implantable cardiac device to meet the requirements of a specific patient, provide technical support for the implantable cardiac device, and troubleshoot problems with the implantable cardiac device, among other purposes. In most circumstances, these device representatives must be present during all stages of care for a patient with an implantable medical device, including, for example, in an operating room, in a catheterization laboratory (cath lab) or even during more routine physician visits.

Generally, each medical device manufacturer also produces a physical device (used by the device representatives) for interacting with its implantable cardiac devices, often referred to as a programmer. These programmers are specific to a particular device manufacturer's cardiac devices. That is, an implantable cardiac device from a specific manufacturer can only be programmed with the manufacturer's specific programmer, and vice versa. Further, each medical device manufacturer's programmer uses its own proprietary graphical interface.

### SUMMARY

Disclosed herein are systems, methods, and devices for providing remote technical support of implantable cardiac devices. In some instances, these devices can eliminate the need for device representatives to be physically present during medical care of a patient having an implantable medical device.

In one embodiment, a device for remotely supporting an implantable cardiac device is disclosed. The device may include at least one video input configured to receive at least a video signal from a programmer for an implantable cardiac device, at least one audio input, a network interface, a display, and/or a user input. The user input can be at least one of a keyboard, mouse, or touchscreen. The device may also include one or more hardware processors configured with instructions to transmit one or more video signals received at the at least one video input over a network to a remote location via the network interface, transmit one or more audio signals received at the at least one audio input over the network to the remote location via the network interface, and/or receive patient data from a database on the network via the network interface, the patient data including at least data uploaded from a home monitoring device, and display the patient data on the display.

In some embodiments, the one or more hardware processors are further configured to transmit the one or more video signals and the one or more audio signals to the remote location via the network interface in real-time or in substantially real-time. The device may comprise one or more additional video inputs. The one or more additional video inputs may be configured to connect to video output of additional devices, such as, medical imagers, cameras, and patient diagnostic equipment, among others. In some embodiments, the at least one video input is a VGA input, although other types of video inputs, such as, DVI, HDMI, SDI, coaxial, etc., are possible. In some embodiments, the one or more hardware processors are further configured with instructions to encrypt the one or more video signals and one or more audio signals transmitted over the network to the remote location via the network interface.

In another embodiment, a method for remotely supporting an implantable cardiac device is disclosed. The method may include connecting a video output of a programmer for an implantable cardiac device to a video input of a proximally located support device. The support device may include a video input configured to receive at least a video signal from a programmer for an implantable cardiac device, a network interface, and one or more hardware processors configured with instructions to transmit a video signal received at the video input over a network to a remote location via the network interface. The method may further include transmitting the video output of the programmer to the remote location in substantially real-time. In some embodiments, the video output of the programmer is analyzed or otherwise used at the remote location to provide remote support for the implantable cardiac device.

In some embodiments, the support device further includes an audio input, and the method further includes receiving an audio signal via the audio input and transmitting the audio signal to the remote location in substantially real-time. The audio signal may be used at the remote location to provide remote support for the implantable cardiac device. In some embodiments, the support device further includes at least one additional video input, and the method further includes connecting a video output of at least one additional device to the at least one additional video input, receiving at least one additional video signal via the at least one additional video input, and transmitting the at least one additional video signal to the remote location in substantially real-time. The one or more additional video inputs may be configured to connect to video output of additional devices, such as, medical imagers, cameras, and patient diagnostic equipment, among others. In some embodiments, the support device further includes a display, and the method further includes receiving patient data from a database on the network via the network interface, the patient device including at least data uploaded from a home monitoring device, and displaying the patient data on the display of the support device. The method may also include transmitting the patient data to the remote location.

In another embodiment, a system for remotely supporting a medical device is disclosed. The system may include, for example, a mobile cart having a tabletop, storage for one or more devices, and wheels. The cart supports a display, a camera, a user interface, and a rechargeable battery. The system also includes a remote support device supported by the cart. The remote support device includes at least one video input configured to receive at least a video signal from a programmer for an implantable cardiac device, a network interface, and one or more hardware processors configured with instructions to transmit one or more video signals received at the at least one video input over a network to a remote location via the network interface.

In some embodiments, the storage for one or more devices comprises one or more shelves. In some embodiments, the storage comprises a cabinet. In some embodiments, the at least one video input and the network interface are positioned on an I/O panel on an exterior surface of the cabinet. In some embodiments, the one or more devices comprise programmers for implantable cardiac devices. In some embodiments, the remote support device includes at least one audio input. In some embodiments, the one or more hardware processors are further configured to transmit one or more audio signals received at the at least one audio input over the network to the remote location via the network interface.

### BRIEF DESCRIPTION OF THE DRAWINGS

The features and advantages of the remote support devices, systems, and methods described herein will become more fully apparent from the following description and appended claims, taken in conjunction with the accompanying drawings. These drawings depict only several embodiments in accordance with the disclosure and are not to be considered limiting of its scope. In the drawings, similar reference numbers or symbols typically identify similar components, unless context dictates otherwise. The drawings may not be drawn to scale.

FIG. 1A shows a perspective view of one embodiment of a device for remotely supporting a medical device.

FIG. 1B shows a simplified block diagram representation of some of the components of the device of FIG. 1A.

FIG. 2 shows a block diagram representing one embodiment of a system for remotely supporting a medical device using a remote support device.

FIG. 3 shows a block diagram of a generalized system for providing remote technical support.

FIG. 4 illustrates one embodiment of a cart including a device for remotely supporting a medical device.

FIG. 5 illustrates another embodiment of a cart including a device for remotely supporting a medical device.

### DETAILED DESCRIPTION

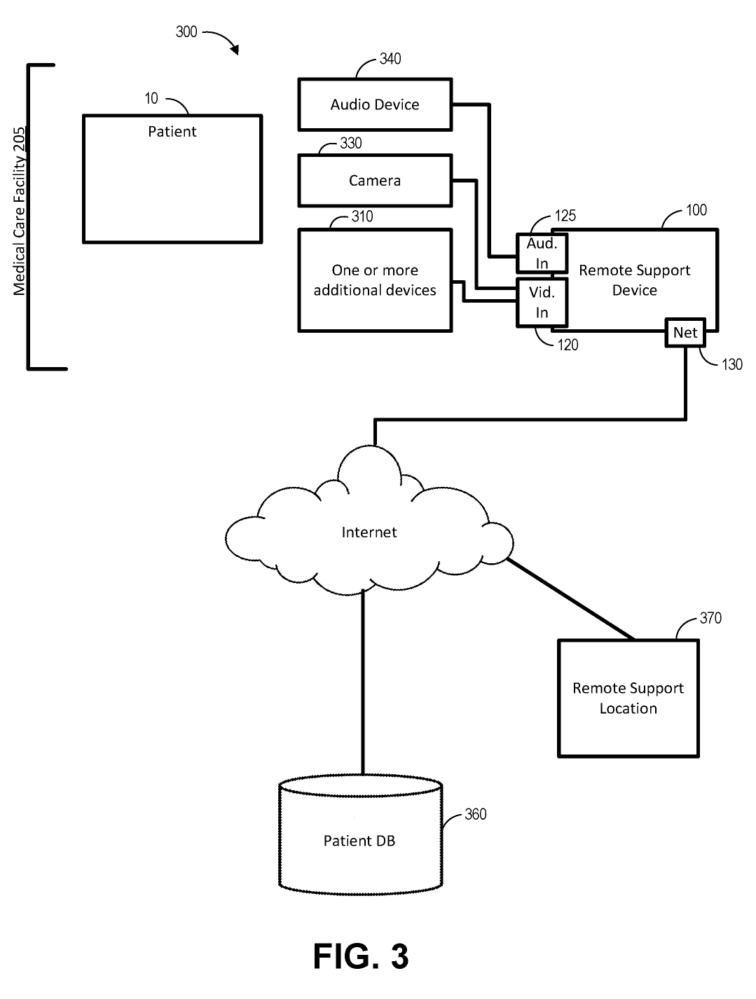

In some embodiments, the systems, methods, and devices described herein provide for remote technical support of medical devices, such as, for example, implantable cardiac devices, and, in some instances, can eliminate the need for device representatives to be physically present during medical care of a patient having an implantable medical device. These and other advantages will become more fully apparent to one of skill in the art upon consideration and review of this disclosure. While this disclosure describes various detailed embodiments, it is not intended to be limited to only the illustrated and described embodiments. For example, while much of the following material discusses providing remote technical support for implantable cardiac devices, this disclosure is not limited to only this example, and the principles disclosed herein may be applied in a generalized form to a wide variety of applications as discussed below in reference to FIG. 3. The disclosed embodiments may be varied, modified, and altered without departing from the scope of the inventions described herein. Further, while many variations are contemplated for different applications and design considerations, for the sake of brevity each and every contemplated variation is not individually described.

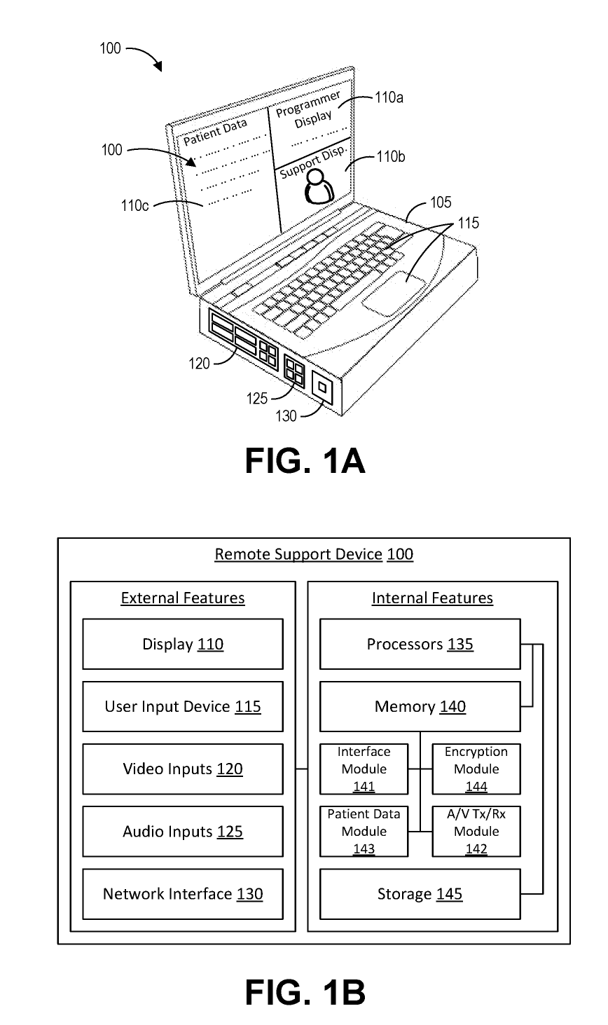

FIGS. 1A and 1B illustrate one embodiment of a device 100 for remotely supporting a medical device, such as, for example, an implantable cardiac device. FIG. 1A shows a perspective view of one embodiment of the device (although other embodiments are possible) and FIG. 1B shows a simplified block diagram representation of some of the components of the device. The device 100, also referred to as the remote support device 100, is useable to allow for remote support of an implantable cardiac device, among other uses. In some embodiments, the device 100 is located proximal to the patient and/or implantable cardiac device and allows support by a remotely located support agent. In some instances, use of the device 100 eliminates the need for a manufacturer's device representative to be physically present with the patient and/or implantable cardiac device. Further, the device 100 may be used to remotely support an implantable cardiac device irrespective of the particular manufacturer of the implantable cardiac device. That is, the device 100 may be used with any manufacturer's implantable cardiac device.

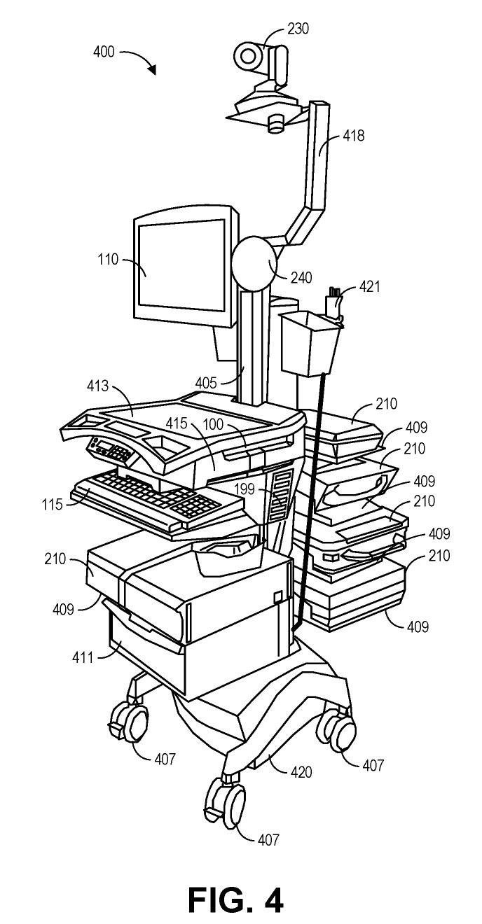

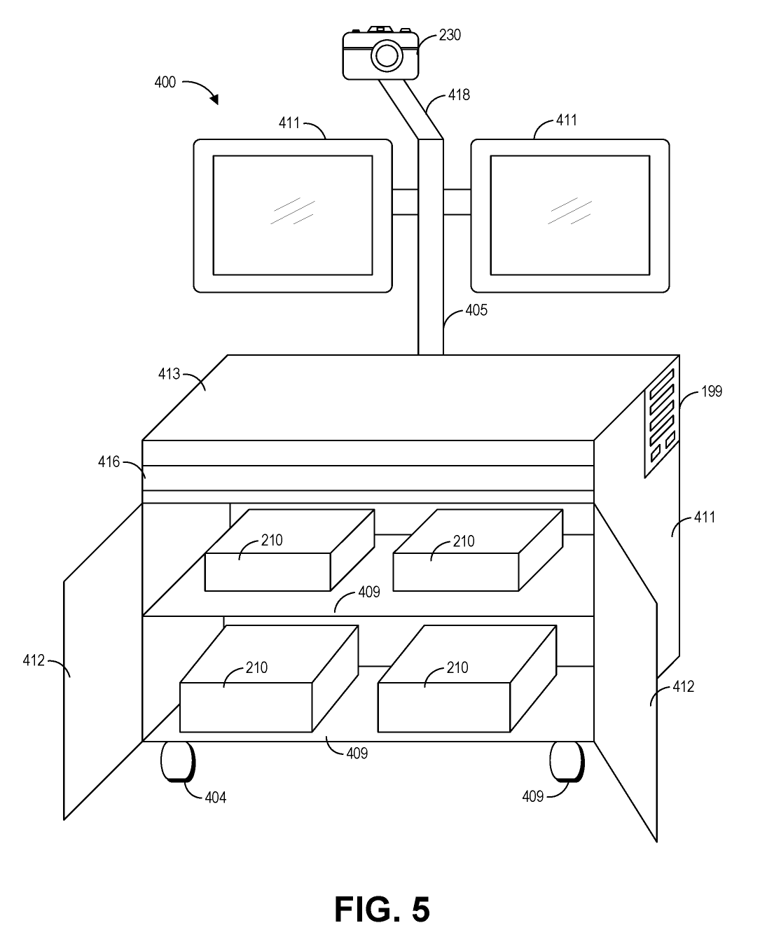

In some embodiments, the components of device 100 are located on or within a common housing 105. In the illustrated embodiment, the housing 105 is illustrated with a form factor that is similar to a laptop. However, the housing 105 need not be limited to this shape. Further, not all of the components of the device 100 need be located on or within a single housing 105. For example, the display 110 and/or the user input devices 115 may be located external to the housing 105. That is, the display 110 and/or the user input devices 115 may be peripheral devices attached to the device 100 via appropriate connectors. The device 100 may also include one or more of the following externally available features or components. These features may include the display 110, the user input devices 115, one or more video inputs 120, one or more audio inputs 125, and/or one or more network interfaces 130. As shown in FIGS. 4-5 below, the device 100 may be included in a cart.

The display 110 is configured to display information to a user. In the illustrated embodiment of FIG. 1A, the display 110 includes three display portions: a programmer display 110a, a support display 110b, and a patient data display 110c. The programmer display 110a may mirror a display of a programmer for an implantable cardiac device that is attached to the remote support device 100. The support display 110b may show information received from a remote support location, such as, a video display of a remote support agent or remote support instructions, among other information. The patient data display 110c may show information related to the patient into which the implantable cardiac device has been implanted. In some embodiments, this patient data includes information uploaded to a remotely located server by a home monitor located in the patient's home. Accordingly, the device 100 can provide patient specific information on the display 110. These display portions 110a, 110b, 110c are provided by way of example only. In some embodiments, one or more of these may be omitted and/or combined with other displays of information. In some embodiments, the display 110 may be omitted.

The device 100 may also include one or more user interface devices 115. For example, in the illustrated embodiment of FIG. 1A, the user interface devices 115 include a keyboard and a track pad. In some embodiments, the display 110 may be a touchscreen that serves as a user interface device. The device 100 may include any type of user interface device including, but not limited to, keyboards, track pads, mice, or touchscreens. The user interface devices 115 allow a healthcare provider to interact with the device 100. In some embodiments, the device 100 may not include a user input device 115.

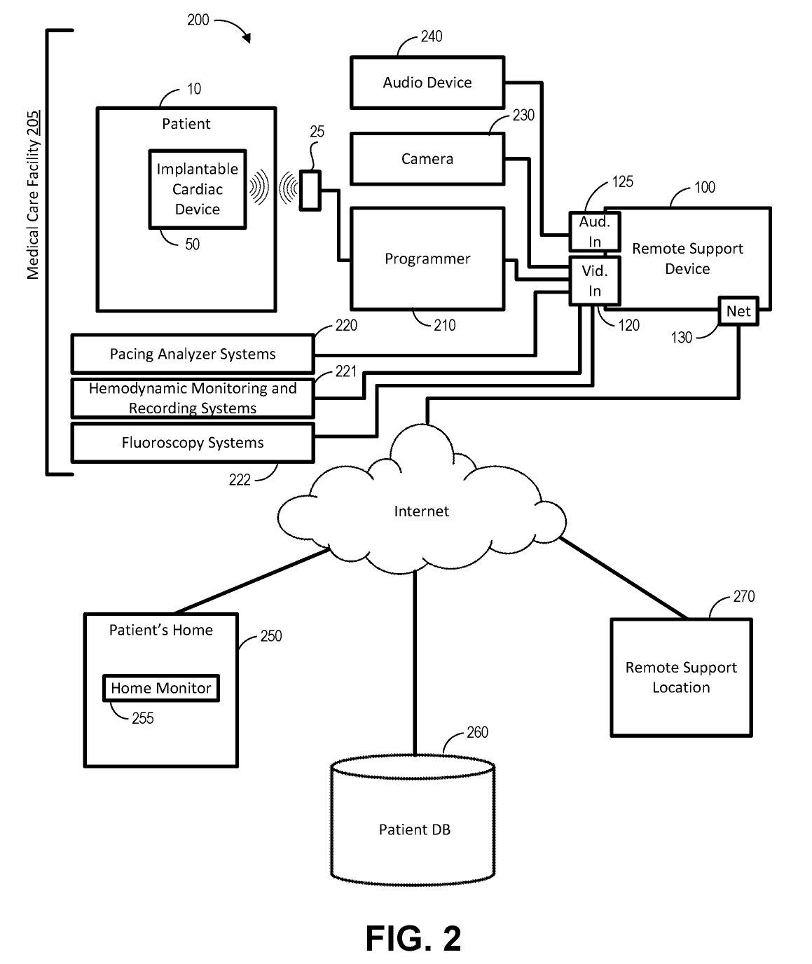

The device 100 also includes one or more video inputs 120. The video inputs 120 are useable to connect the device 100 to the video outputs of one or more additional devices, including, for example, a programmer 210 for an implantable cardiac device, a pacing system analyzer 220, hemodynamic monitoring and recording systems 221 (or other types of patient monitoring system), and a fluoroscopy system 222 (or other type of medical imager system), and/or camera 230, among others (as shown, for example, in FIG. 2). In some embodiments, the video inputs 120 are VGA inputs, DVI inputs, HDMI inputs, SDI inputs, coaxial inputs, display inputs, FireWire inputs, component video inputs (for example, RCA inputs), any other type of video input, or any combination thereof. As just one example, the video inputs 120 can include four VGA inputs, two DVI inputs, two HDMI inputs, and two USB inputs. In some embodiments, the device 100 can include up to ten, or more, video inputs.

The device 100 may also include one or more audio inputs 125. The audio inputs 125 are useable to connect the device 100 to an audio output of an audio capture device, such as a microphone. In some embodiments, the audio inputs 125 may also be connected to an audio output device, such as a speaker. In some embodiments, the one or more audio inputs 125 can include auxiliary inputs, DIN inputs, XLR inputs, RCA inputs, USB inputs, green audio inputs, pink microphone inputs, any other type of audio input, or any combination thereof. In some embodiments, the device 100 may include up to four, or more, audio inputs. In some embodiments, the device 100 may not include an audio input 125. In some embodiments, the audio inputs 125 can be used to connect the device 100 to a headset, including a speaker and a microphone, or a standalone speaker with a microphone. In some embodiments, the headset may be a Bluetooth headset which connects via a Bluetooth dongle. Use of the dongle may allow a strong connection between the headset and the device 100. In some instances, the headset is a Jabra Evolve 65. This may permit a range of up to 100 meters. This may also permit increased efficiency of communication with computers.

The device 100 also includes a network interface 130. The network interface 130 is useable to connect the device 100 to a network, such as a local area network (LAN) or wide area network (WAN), such as the internet. In some embodiments, the network interface 130 may be wired. In some embodiments, the network interface 130 may be wireless. The network interface 130 allows the device 100 to communicate over the network with other devices as will be described below.

The device 100 may also include one or more of the following internal features/components (in other words, these components are generally located within the housing 105): one or more hardware processors 135, one or more memories 140, and one or more storage devices 145.

Each of the one or more processors 135 may be a central processing unit (CPU) or other type of hardware processor, such as a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, or in the alternative, the processor may be any conventional processor, controller, microcontroller, or state machine. A processor may also be implemented as a combination of computing devices, for example, a combination of a DSP and a microprocessor, a plurality of microprocessors, one or more microprocessors in conjunction with a DSP core, or any other such configuration. The one or more processors 135 may perform logical and arithmetic operations based on program instructions or modules stored within the memories 140 and/or storage devices 145.

The memories 140 and/or storage devices 145 may each be a RAM memory, a flash memory, a ROM memory, an EPROM memory, an EEPROM memory, a register, a hard disk, a removable disk, a CD-ROM, or any other form of storage medium known in the art. Exemplary memories 140 and storage devices 145 are coupled to the one or more processors 135 such that the processors can read information from and write information to the memories 140 and/or storage devices 145. In some embodiments, the memories 140 and/or storage devices 145 may be integral to the processors 135. The memories 140 and/or storage devices 145 can store an operating system that provides computer program instructions for use by the processors 135 or other elements included in the device in the general administration and operation of the device.

In the illustrated embodiment of FIG. 1B, four modules are shown: an interface module 141, an audio/video transmission (A/V Tx/Rx) module 142, a patient data module 143, and an encryption module 144. However, these are provided by way of example only, and may be omitted, combined, and/or modified in a wide variety of ways. The modules described herein may be implemented in hardware, software, or a combination thereof. The modules are executed by the processor, alone or in combination, to operate the device 100.

The interface module 141 may include instructions that configure the processors 135 to operate the display 110 and receive input from the user interface devices 105. For example, the interface module 141 may be configured such that a graphical user interface (GUI) is shown on the display 110. In some embodiments, the GUI may be the same, regardless of the particular implantable cardiac device being supported. That is, the GUI is not dependent on the specific manufacturer of the implantable cardiac device. This may allow the device 100 to be used with implantable cardiac devices irrespective of manufacturer. In some embodiments, the interface module 141 may include instructions that configure the processors 135 to cause the display 110 to display the video signals received at the video inputs 120. For example, in some embodiments, the interface module 141 may cause the display 110 to display video received from the programmer 210, the pacing system analyzer 220, the hemodynamic monitoring and recording systems 221, and/or the fluoroscopy system 222 shown in FIG. 2. In some embodiments, the interface module 141 may include instructions that configured the processors 135 to cause the display 110 to display video signals received from a remote site (such as the remote support location 270) over the network. For example, in some embodiments, the interface module 141 may cause the display 110 to display a video feed of a remote support technician.

The audio/video transmission module 142 may include instructions that configure the processors 135 to send and receive video and/or audio signals to and from the device 100. For example, a video input 120 of the device 100 may be connected to a video output of the programmer 210, the pacing system analyzer 220, hemodynamic monitoring and recording systems 221, and/or the fluoroscopy system 222 shown in FIG. 2. The audio/video transmission module 142 may be configured with instructions that cause the processors 135 to transmit the video signal received from the programmer 210, the pacing system analyzer 220, hemodynamic monitoring and recording systems 221, and/or the fluoroscopy system 222 over a network via the network interface 130. For example, the video signal received from these device may be transmitted by the audio/video transmission module 142 to the remote support location 270 shown in FIG. 2, where these video signals can be viewed by a remote support technician. In some embodiments, transmission may occur in substantially real time. In some embodiments, transmission may be substantially instantaneous. This may allow the support agent to remotely support the implantable cardiac device. As will be described below, the video outputs of other devices can also be connected to and transmitted through the device 100. Similarly, audio signals received at audio inputs 125 may be transmitted over the network to the support agent. For example, an audio output of a microphone may be connected to the audio inputs 125. This signal may be transmitted over the network such that it can be heard by the remotely located support agent. In some embodiments, the audio/video transmission module 142 works with the interface module 141 to display a video transmitted from remote support location 270 on the display 110. In some embodiments, the audio/video transmission module 142 works with the encryption module 144 (described below) to protect patient data and other transmitted information as required by the Department of Health and Human Services under The Health Insurance Portability and Accountability Act (HIPAA).

The patient data module 143 may include instructions that configure the processors 135 to send and receive patient specific data. In some embodiments, this patient data includes information uploaded to a remotely located server by a home monitor 255 located in the patient's home 250 as shown in FIG. 2. In some embodiments, this patient specific information may be remotely stored in a patient database on a server 260 accessible over the network. The patent data module can work with the audio/video transmission module 142 and the interface module 141 to send and receive patient information over the network and display it on the display 110 and/or at the remote support location 270.

The encryption module 144 may include instructions that configure the processors 135 to encrypt and decrypt all data sent to and from the device 100. In some embodiments, the encryption may be sufficient such that the device 100 is compliant with the Health Insurance Portability and Accountability Act (HIPAA). The encryption module 144 may protect sensitive patient data. For example, in some embodiments, patient data is encrypted as it is transmitted between the device 100, the patient's home 250, the patient database 260, and/or the remote support location 270. As another example, in some embodiments, the video signals of the programmer 210, the pacing system analyzer 220, the hemodynamic monitoring and recording systems 221, and/or the fluoroscopy system 222 are encrypted for secure transmission by the audio/video transmission module 142 over the network from the device 100 to the remote support location. Accordingly, the patient data module 143 works with the other modules described above to protect information sent to and from the device 100. As noted above, encryption module 144 may be configured for HIPAA compliance.

In some embodiments, the remote support device 100 includes an alarm or alert that can be remotely activated by a remote support technician at the remote support location 270. The alarm or alert may include a visual alarm or alert (e.g., a flashing light) and/or an audible alarm (e.g., a siren). The alarm or alert may be activated by a remote support technician to draw attention to healthcare providers located in proximity to the remote support device 100 and the patient. For example, if a remote support technician detects an emergency condition, the remote support technician may activate the alarm or alert to notify healthcare providers. In some embodiments, the remote support technician may be able to page healthcare providers.

The device 100, may be embodied in a variety of different ways. For example, FIG. 1A illustrates the device 100 as disposed within common housing and having a form factor that may be similar, in some respects, to a traditional laptop. However, other configurations are possible. For example, the device 100 may be embodied as one or more computer components positioned on a cart (for example, as shown in FIGS. 4-5 and described below).

FIG. 2 shows a block diagram representing one embodiment of a system for remotely supporting an implantable cardiac device 50 using a remote support device 100. The remote support device 100 may include the features and components previously discussed in reference to FIGS. 1A and 1B. The remote support device can be included in a cart-based system as shown in FIGS. 4 and 5. The system 200 may be used such that an implantable cardiac device 50 implanted (or being implanted) in a patient 10 in a medical healthcare facility 205 can be remotely supported by a technician at a remote support location 270. Thus, with the system 200, device representatives need not be physically present to provide support for implantable cardiac device 50.

In the illustrated embodiment, the system 200 includes a remote support device 100 that is connected to a programmer 210 (which, in some embodiments, can include an EKG device), a pacing system analyzer system 220, a hemodynamic monitoring and recording system 221 (or other type of patient monitoring system), and a fluoroscopy device 222 (or other type of medical imaging device), a camera 230, and an audio device 240. However, the system 200 is not limited to the illustrated embodiment. For example, the remote support device 100 may be connected to more, fewer, or different devices than are illustrated in FIG. 2. The remote support device 100 is further connected to a network, such as the internet. In some embodiments, the remote support device 100 is connected to a private LAN, through which it has access to the internet. The remote support device 100 is configured, as described above, to receive video signals via one or more video inputs 120 and/or audio signals via one or more audio inputs 125 and transmit them, in substantially real time, over the network, via network interface 130, to remote support location 270.

In some embodiments, the remote support device 100 may capture one or more analog VGA signals (or other types of video or audio signals), encode, encrypt the signals, and then transmit the signals to the remote support location 270 using a UDP protocol (or other type of transmission protocol). UDP may be used because, in some embodiments, it can minimize the latency of the video/audio stream and provide the most bandwidth savings for both the medical care facility 205 and remote support location 270. In some embodiments, each remote support device 100 has a secure SSL/TLS administration and control tunnel to the remote support location 270. The tunnel may be established via TCP protocol and may be used for stream initiation and completion, remote control and administration of the remote support device 1100 by the support technicians at the remote support location 270. In some embodiments, the SSL/TLS tunnel might also work as a backup media to transmitting the video streaming case the medical care facility 205 will only allow one outbound TCP port opened on the firewall. However, this may result in increased latency.

The programmer 210 is typically a manufacturer specific hardware device configured to interface with an implantable cardiac device 50 for programming. The programmer 210 may include a wand or donut 25, which can be positioned proximal to the implantable cardiac device 50 and establish wireless communication with the implantable cardiac device 50. Thus, the programmer 210 is able to communicate with the implantable cardiac device 50 even when the implantable cardiac device is surgically installed in the patient's 10 body. In general, each programmer 210 has a manufacturer specific interface and is only able to program a specific manufacturer's implantable cardiac devices. Thus, the system 200 may include a plurality of programmers 210 for each of the different medical device manufacturers. The programmer 210 includes a video output. The video output of the programmer 210 is connected to a video input 120 of the remote support device 100. Thus, the video output of the programmer 210 can be transmitted to the remote support location 270 by the remote support device 100, such that remote technicians can view the output of the programmer 210.

The system 200 may include a fluoroscopy device 222 (or any other type of medical imager, including but not limited to magnetic resonance imaging machines (MRI), computed tomography machines (CT), conventional X-ray machines, etc.) In some embodiments, the fluoroscopy device 222 (or other medical imager) can be located in a cath lab. Each fluoroscopy device 222 (or other medical imager) includes a video output that can be connected to the video input 120 of the remote support device 100. Thus, the video output of the fluoroscopy device (or potentially some of or each of the fluoroscopy devices) 222 (or other medical imager(s)) can be transmitted to the remote support location 270 by the remote support device 100, such that remote technicians can view the output of the fluoroscopy device 222 (or other medical imager). The system 200 may also include one or more hemodynamic monitoring and recording systems 221 (or other type of patient monitor) connected to the device 100. Hemodynamic monitoring and recording systems 221 (or other type of patient monitor) may be any type of medical device that measures and displays information about the patient. For example, such devices can include a heart rate monitor, blood pressure monitor, blood oxygenation monitor, etc. In general, hemodynamic monitoring and recording systems 221 (or other type of patient monitors) may include displays for showing their measured parameters in the medical care facility. The hemodynamic monitoring and recording systems 221 (or other type of patient monitors) may also include video outputs that are connected to the device 100. In this way, the device 100 can receive the video output from the hemodynamic monitoring and recording systems 221 (or other type of patient monitors) and the video output of these devices may be remotely visible to the remote technicians. The system 200 may also include a pacing analyzer system 220. In some embodiments, the pacing analyzer system 220 can be combined with the programmer 210. In some embodiments, the system 200 may also include an EKG, ECG, or any number of additional medical devices. Each of these devices may include a video output connected to the device 100. The device 100 may thus transmit the video output of these devices to the remote technicians. Any type of device with a video output may be connected to the device 100 to relay the video output date to the remote technicians.

System 200 may also include one or more cameras 230. The cameras 230 may capture video or still images. In some embodiments, at least one of the cameras 230 is moveable by persons located at the medical facility 205. The cameras 230 include a video output that can be connected to the video input 120 of the remote support device 100. Thus, the video output of each of the cameras 230 can be transmitted to the remote support location 270 by the remote support device 100, such that remote technicians can view the output of the cameras 230. This allows the remote support technicians a real-time view of the medical care facility 205.

System 200 may also include an audio device 240. For example, audio device 240 may be a microphone and/or speaker. In some embodiments, the audio device may include a headset (with a microphone and speaker). In some embodiments, the device 100 may include Bluetooth, such that a connection between the device 100 and the audio device 240 is made over Bluetooth. The audio device 240 may capture and/or emit sound. The audio device 240 includes an audio output that can be connected to the audio input 125 of the remote support device 100. Thus, the remote support device 100 can transmit audio from the medical care facility 205 over the network to the remote support location 270. In some embodiments, audio device 240 allows for two-way communication such that technicians at the remote support location 270 can communicate in real time with healthcare providers at the healthcare facility 205. In some embodiments, audio communication in the system 200 need not pass through remote support device 100. For example, audio communication may occur over traditional telephone or VOIP technologies.

The remote support location 270 is also connected to the internet, and thus can be located anywhere and still be able to communicate with the remote support device 100. Support technicians at the remote support location are able to view the outputs of the programmer 210, the pacing analyzer systems 220, the hemodynamic monitoring and recording systems 221, the fluoroscopy systems 222, and camera 230 and hear the audio output of the audio device 240 such that they can provide support for the implantable cardiac device 50, even though they are remotely located. The remote support device 100 allows the technicians to support the implantable cardiac device 50 as if they were present in the medical care facility 205.

In some embodiments, an agent interface is available to the support technicians at remote support location 270. The agent interface may be available on the support technician's workstation (for example, computer) and can provide the capability to view the appropriate video and/or audio streams. The interface may be easy to use and intuitive. Under normal circumstances, as soon as a call is received and the calling party information is available, the interface may start automatically and display the proper video stream. If the calling party information is unavailable due to unforeseen circumstances, the support technician still may have an option of initiating the appropriate video stream and displaying it via the interface. The support technician may be presented with a list of hospitals and available remote support devices to connect to, from which he can make a selection. Once the video viewing is no longer needed the support technician may be able to issue a stop control to the remote support device. Furthermore, the agent may be able to start and stop or view any video stream from any remote support device at any time, without a phone call from the hospital or after the call has been disconnected. In some embodiments, calls may only be initiated and/or terminated from the remote support location 270. For example, in some embodiments, an end user (e.g., a nurse located with the remote support device 100) does not have control over a call session, except to activate the audio and video feeds. That is, in some embodiments, the end user cannot initiate a call, but most contact the remote support location 270 in order to initiate a call. Thus, in some embodiments, control of the remote support device 100 can be described as “one-way,” and can only be controlled from the remote support location 270. In some embodiments, one-way control of the remote support device 100 improves security.

System 200 may also include home monitors 255 located in the patients' homes 250 and a patient database 260, all of which can be connected to the internet. In general, patients having an implantable cardiac device have a home monitor 255 in their home 250 that communicates wirelessly with the implantable cardiac device. The home monitor 255 gathers information and other usage statistics from the implantable cardiac device. This information can be uploaded from the home monitor 255 to the patient database 260 over the internet. This information may then be accessible in the medical care facility 205 via the remote support device 100 and at the remote support location 270. Having this patient information available during support of the implantable cardiac device 50 greatly improves the technicians' and health care providers' ability to support the implantable cardiac device and provide care to the patient. In some embodiments, this patient information can be sent from the patient database 260 to the remote support device 100 and displayed on the display 110 of the remote support device 100.

Although the description of system 200 focused primarily on an example of providing remote technical support for an implantable cardiac device, the principles of this disclosure may be generalized and applied in a wide variety of applications. FIG. 3 illustrates a system 300 that utilizes an embodiment of a device 100, as described above, in a generalized remote support application.

As shown in FIG. 3, the remote support device 100 may be located at a medical care facility proximally positioned to a patient 10. The remote support device 100 may be connected to an audio device 340, a camera 330, and one or more additional devices 310. The device 100 relays the output signals of each of these devices over the internet to a remote support location 370. Remote support technicians at the remote support location 370 are able to view the output signals of each of the audio devices 340, the camera 330, and the one or more additional devices 310. In this way, they are able to provide remote technical support, having access to the same information that is available at the medical care facility. The audio device 340 and camera 330 allow the remote support technicians to view and hear the patient and situation in the medical care facility in real time. The one or more additional devices 310 may be any type of device that includes a video output, including all types of medical imaging devices, fluoroscopy devices, patient monitoring devices, etc. Thus, the remote support technicians can view the video output of any additional devices displaying data at the medical care facility. Patient data may be stored at a patient database 360 accessible over the network and accessible at remote support location 370, medical care facility, or both.

The system 300 can be used in a wide variety of applications. For example, it can be used during electrophysiology (EP) procedures, which include EP studies and cardiac ablations, as well as the orthopedic procedures (spine and joints) and general surgeries, among others. In any of these situations, the device 100 allows a remotely located support technician to view the situation substantially as if present. The system 300 can also be used, for example, in peer-to-peer medical training situations. For example, a less experienced doctor may use the system 300 to consult with a more experienced doctor. The system 300 allows the more experienced doctor to view the situation as if present with the less experienced doctor.

In some embodiments, the remote support device 100 and system 200 provide a fully automated video transmitting system that requires virtually no maintenance. In some embodiments, the remote support device 100 and system 200 are remotely controlled by remote support technicians at the remote support location 270. The remote support device 100 and system 200 may provide the ability to see video streams in parallel with voice conversation. In some embodiments, the remote support device can transmit video with a resolution of 1024×768 at 10-15 frames per second, 1920×1080 at 30 frames per second, or more. In some embodiments, the remote support device 100 and system 200 can make use of existing network LAN/WAN infrastructure. In some embodiments, the remote support device 100 and system 200 provide a simple to use platform with web-based configuration and viewer. In some embodiments, the remote support device 100 can be highly mobile and portable. Further, the remote support device 100 can be easy to assemble, disassemble, move and store while not in use and may not require any configuration changes if moved from one room to another within the same LAN. In some embodiments, the remote support device 100 dissipates less than 15 watts of heat using passive cooling and has no moving parts so that it is completely silent.

In some embodiments, remote administration of the remote support devices 100 such as firmware upgrades and other troubleshooting can be done via a built in web interface. A technician will be able to connect to a web-based management interface from the remote support location. The web interface is also accessible from local LAN, such as from within the medical care facility 205.

In some embodiments, the remote support device 100 uses a UDP video stream that is encrypted at the source on the remote support device 100 device using a Blowfish encryption algorithm (based on OpenSSL). Other methods of encryption may also be used. The key exchange between the remote support device 100 and the remote support location 270 may be done through the SSL/TLS administration and control tunnel. In some embodiments, a TCP administration and control tunnel between the remote support device 100 and the remote support location 270 is secured using SSL/TLS technology. All communication between the remote support device 100 and the remote support location 270 may go through the administration and control tunnel, including encryption key exchange, stream control, and the web administration interface. All web interfaces may be authenticated with a username/password combination and protected by HTTPS. In some embodiments, the remote support device 100 uses a two-factor authentication.

FIG. 4 illustrates one embodiment of a cart 400 including a device 100 for remotely supporting a medical device. In the illustrated embodiment, the cart 400 includes a frame 405 supporting one or more components placed thereon. The frame 405 includes wheels 407 such that the cart 400 is mobile. The frame 405 may also include one or more shelves 409 and storage compartments 411. The shelves 409 and storage compartments 411 may be used to support or store various components (such as components of the systems 200 and 300 of FIGS. 2 and 3) as described below.

In the illustrated embodiment, the cart 400 also includes an enclosure 415 that includes a remote support device 100. The remote support device 100 may be similar to any of the remote support devices 100 described above with reference to FIGS. 1-3. In some embodiments, the remote support device 100 is integrated with the cart 400. In some embodiments, the remote support device 100 is removable from the cart 400. The remote support device 100 includes an input/output panel (“I/O panel”) 199 that includes video inputs 120, audio inputs 125, and/or network interfaces 130 as described above. In the illustrated embodiment, the I/O panel 199 is located on the cart 400 such that it is externally accessible, such that the video and/or audio outputs of additional devices may be connected thereto. In some embodiments, the audio inputs 125 can be located on the speaker 240. As described above, video and/or audio outputs of additional devices may be connected to the remote support device 100 and the remote support device 100 may be configured to transmit these signals to a remote support technician in a remote location.

The remote support device 100 may also be connected to one or more displays 110 and/or inputs 115 (such as the keyboard and mouse illustrated in FIG. 4) that are supported by the cart 400. In some embodiments, more than one display 110 may be included. For example, two displays 110 may be included on the cart 400. Other types of inputs 115 (in addition to or in place of the illustrated keyboard and mouse) may also be incorporated into the cart 400.

The cart 400 may also include a camera 230 that is connected to the remote support device 100. The remote support device 100 may be configured to transmit video and/or still images received from the camera 230 to a remote support technician as described above. The camera 230 may be supported on an arm 418 that extends from the cart 400. The arm 418 may be configured to articulate such that the position and view of the camera 230 can be adjusted. In some embodiments, the cart 400 is positioned such that the arm 418 and camera 230 can be remotely controlled such that a remote support technician can remotely adjust the position and view of the arm 418 and camera 230. In some embodiments, the cart 400 also includes a wireless camera (not illustrated) that can be removed from the cart and positioned proximal to (for example, above) a patient. Images and video from the wireless camera can be transmitted to a remote support technician via the remote support device 100.

The cart 400 may also include an audio device 240. The audio device 240 may be a speaker and/or a microphone.

In some embodiments, the cart 400 includes a battery 420 configured to provide power to the cart 400, the remote support device 100, and/or other components (such as the programmers 210 described below). The battery 420 may be rechargeable. The cart 400 may include a power cable 421 for charging the battery 420. In some embodiments, the battery 420 may be omitted and the power cable 421 may supply power for the cart 400 and other components directly.

In some embodiments, the cart 400 also includes a visual alert (not shown) such as a light that can be used to provide visual cues, alarms, or warnings. For example, the visual alert may be configured to light up when patient vital signs indicate an emergency condition. In some embodiments, the remote support technician can remotely trigger the visual alert. Similarly, in some embodiments, the cart 400 may include an audible alert (such as a siren).

The shelves 409 may be configured to store or support various components used in conjunction with the cart 400. For example, in some embodiments, the shelves 409 are configured to support various programmers 210 (such as the programmers 210 described above with reference to FIGS. 2-3). The programmers 210 may be configured to interface with various medical devices, such as, for example, implantable cardiac devices. The cart 400 may be configured to store a plurality of programmers 210 corresponding to the programmers 210 of a plurality of implantable cardiac device manufacturers. Thus, the cart 400 may be used to support a plurality of implantable cardiac devices by selecting the appropriate programmer 210 depending on the implantable cardiac device to be remotely supported.

In some embodiments, in use, a programmer 210 is selected that corresponds with the implantable cardiac device to be supported. The selected programmer 210 is removed from the shelf 409 and placed on a table top 413 of the cart 400. The video output of the selected programmer 210 is then connected to the I/O panel 199 of the remote support device 100 so that the video output can be transmitted to a remote support technician. In some embodiments, various cables and connectors necessary to connect the selected programmer 210 to the remote support device are conveniently stored in the storage compartment 411, which, as illustrated, may be a drawer attached to the cart 400. Other devices, such as pacing analyzer systems 220, hemodynamic monitoring and recording systems 221, fluoroscopy systems 222, or other devices 310 shown in FIGS. 2 and 3 may also be connected to the I/O panel 199 of the remote support device 100, and the signals of these devices may also be transmitted to the remote support technician. Thus the cart 400 may be used in the systems 200 and 300 of FIGS. 2 and 3 for providing remote technical support.

While one embodiment of a cart 400 is shown in FIG. 4, other embodiments are possible. For example, the size and number of components included on the cart 400 may be increased or decreased depending on the particular application. The cart 400 illustrated in FIG. 4 is one embodiment of a configuration that can be used to support implanted cardiac devices, for example in a cath lab. As such, this embodiment includes one or more programmers 210. However, the cart 400 may be used to provide other types of support, and as such may include different components in some embodiments.

For example, in another embodiment, the cart 400 may be configured for use in a physician's office. The cart 400 configured for use in a physician's office may include fewer components than the cart 400 configured for use in a cath lab. For example, physician's office cart 400 may not include the programmers 210.

In some embodiments, the cart 400 including a remote support device 100 can be configured for use in a hospital, such as in an operating room or cath lab. The cart 400 provides a form factor that is conducive to this environment, as it provides all the necessary equipment on a mobile platform. In some embodiments, a standalone remote support device 100, for example, with the form factor illustrated in FIG. 1A, may be configured for use in a physician's office. This form factor may provide advantages in a physician's office where less equipment is needed. The remote support device 100 may also be used in a hospital for non-surgical operations, for example, in addition to the operating room, the remote support device 100 can be used in an emergency room or during follow ups in a hospital, among other uses.

FIG. 5 illustrates another embodiment of a cart 400 including a device for remotely supporting a medical device. The cart 400 illustrated in FIG. 5 is similar to the cart 400 illustrated in FIG. 4, except that the cart 400 of FIG. 5 is configured with an enclosure 411 for enclosing the programmers 210 and/or other devices. The enclosure 411 may be a cabinet with doors 412 that includes shelves 419 for supporting the programmers 210 and/or other devices. A remote support device 100 (not shown) may also be housed within the enclosure and an I/O panel 199 of the remote support device 100 may extend through the enclosure 411 to be externally accessible. The enclosure 411 may also include an extendable tray 416 that can support input devices, such as a mouse and keyboard, among others. The top of the enclosure 411 forms a table top 413 on which a selected programmer may be placed during use. In the illustrated embodiment, the cart also includes wheel 407, two displays 411 supported by a frame 405, and a camera 230 supported by an arm 418. The cart 400 of FIG. 5 may also include any of the features of the cart 400 of FIG. 4 described above.

Although this disclosure has primarily discussed providing remote support for implantable cardiac devices, it is recognized that the systems, devices, and methods disclosed herein have application in other areas. For example, systems, devices, and methods disclosed herein may be adapted for use with other implantable (or non-implantable or external) medical devices that are not used with the heart. Thus, this disclosure need not be limited to only cardiac devices, but may apply generally to all types of medical devices. Further, this disclosure has applicability outside of the field of healthcare. For example, this disclosure may be adapted to provide remote technical support for all types of electrical and/or mechanical devices.

The foregoing description details certain embodiments of the systems, devices, and methods disclosed herein. It will be appreciated, however, that no matter how detailed the foregoing appears in text, the systems, devices, and methods can be practiced in many ways. As is also stated above, it should be noted that the use of particular terminology when describing certain features or aspects of the invention should not be taken to imply that the terminology is being redefined herein to be restricted to including any specific characteristics of the features or aspects of the technology with which that terminology is associated.

It will be appreciated by those skilled in the art that various modifications and changes may be made without departing from the scope of the described technology. Such modifications and changes are intended to fall within the scope of the embodiments. It will also be appreciated by those of skill in the art that parts included in one embodiment are interchangeable with other embodiments; one or more parts from a depicted embodiment can be included with other depicted embodiments in any combination. For example, any of the various components described herein and/or depicted in the Figures may be combined, interchanged or excluded from other embodiments.

As used herein a graphical user interface may include a web-based interface including data fields for receiving input signals or providing electronic information. The graphical user interface may be implemented in whole or in part using technologies such as HTML, Flash, Java, .net, web services, and RSS. In some implementations, the graphical user interface may be included in a stand-alone client (for example, thick client, fat client) configured to communicate in accordance with one or more of the aspects described.

The various operations of methods described above may be performed by any suitable means capable of performing the operations, such as various hardware and/or software component(s), circuits, and/or module(s). Generally, any operations illustrated in the Figures may be performed by corresponding functional means capable of performing the operations.

Information and signals may be represented using any of a variety of different technologies and techniques. For example, data, instructions, commands, information, signals, bits, symbols, and chips that may be referenced throughout the above description may be represented by voltages, currents, electromagnetic waves, electromagnetic fields or particles, optical fields or particles, or any combination thereof.

The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. The described functionality may be implemented in varying ways for each particular application, but such implementation decisions should not be interpreted as causing a departure from the scope of the embodiments of the disclosure.

The various illustrative blocks, modules, and circuits described in connection with the embodiments disclosed herein may be implemented or performed with a general purpose processor, a Digital Signal Processor (DSP), an Application Specific Integrated Circuit (ASIC), a Field Programmable Gate Array (FPGA) or other programmable logic device, discrete gate or transistor logic, discrete hardware components, or any combination thereof designed to perform the functions described herein. A general purpose processor may be a microprocessor, but in the alternative, the processor may be any processor, controller, microcontroller, or state machine. A processor may also be implemented as a combination of computing devices, for example, a combination of a DSP and a microprocessor or a plurality of microprocessors, in conjunction with a DSP core, or any other such configuration.

The steps of a method or algorithm and functions described in connection with the embodiments disclosed herein may be embodied directly in hardware, in a software module executed by a processor, or in a combination of the two. If implemented in software, the functions may be stored on or transmitted over as an instruction, instructions or code on a tangible, non-transitory computer-readable medium. A software module may reside in Random Access Memory (RAM), flash memory, Read Only Memory (ROM), Electrically Programmable ROM (EPROM), Electrically Erasable Programmable ROM (EEPROM), registers, hard disk, a removable disk, a CD ROM, or any other form of storage medium known in the art. A storage medium is coupled to the processor such that the processor can read information from, and write information to, the storage medium. In the alternative, the storage medium may be integral to the processor. Disk and disc, as used herein, includes compact disc (CD), laser disc, optical disc, digital versatile disc (DVD), floppy disk and blu ray disc where disks usually reproduce data magnetically, while discs reproduce data optically with lasers. Combinations of the above should also be included within the scope of computer readable media. The processor and the storage medium may reside in an ASIC. The ASIC may reside in a user terminal. In the alternative, the processor and the storage medium may reside as discrete components in a user terminal.

Information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

For purposes of summarizing the disclosure, certain aspects, advantages and novel features have been described herein. It is to be understood that not necessarily all such advantages may be achieved in accordance with any particular embodiment. Thus, the invention may be embodied or carried out in a manner that achieves or optimizes one advantage or group of advantages as taught herein without necessarily achieving other advantages as may be taught or suggested herein.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention as embodied in the attached claims. Applicant reserves the right to submit claims directed to combinations and sub-combinations of the disclosed inventions that are believed to be novel and non-obvious. Inventions embodied in other combinations and sub-combinations of features, functions, elements and/or properties may be claimed through amendment of those claims or presentation of new claims in the present application or in a related application. Such amended or new claims, whether they are directed to the same invention or a different invention and whether they are different, broader, narrower or equal in scope to the original claims, are to be considered within the subject matter of the inventions described herein.

What is claimed is:

1. A device for remotely supporting an implantable cardiac device, the device comprising:
at least one video input configured to receive at least a video signal from a programmer for an implantable cardiac device;
at least one audio input;
a network interface;
a display;
a user input, including at least one of a keyboard, mouse, or touchscreen; and
one or more hardware processors configured with instructions to:
transmit one or more video signals received at the at least one video input over a network to a remote location via the network interface;
transmit one or more audio signals received at the at least one audio input over the network to the remote location via the network interface;
receive patient data from a database on the network via the network interface, the patient device including at least data uploaded from a home monitoring device; and
display the patient data on the display.

2. The device of claim 1, wherein the one or more hardware processors are further configured to transmit the one or more video signals and the one or more audio signals to the remote location via the network interface in substantially real-time.

3. The device of claim 1, further comprising one or more additional video inputs.

4. The device of claim 1, wherein the at least one video input is a VGA input.

5. The device of claim 1, wherein the one or more hardware processors are further configured with instructions to encrypt the one or more video signals and one or more audio signals transmitted over the network to the remote location via the network interface.

6. A method for remotely supporting an implantable cardiac device, the method comprising:
connecting a video output of a programmer for an implantable cardiac device to a video input of a proximally-located support device, the support device comprising:
a video input configured to receive at least a video signal from a programmer for an implantable cardiac device,
a network interface, and
one or more hardware processors configured with instructions to transmit a video signal received at the video input over a network to a remote location via the network interface;
transmitting the video output of the programmer to the remote location in substantially real-time.

7. The method of claim 6, wherein the support device further comprises an audio input, and wherein the method further comprises receiving an audio signal via the audio input and transmitting the audio signal to the remote location in substantially real-time.

8. The method of claim 6, wherein the support device further comprises at least one additional video input, and wherein the method further comprises:
connecting a video output of at least one additional device to the at least on additional video input;
receiving at least one additional video signal via the at least one additional video input;
and transmitting the at least one additional video signal to the remote location in substantially real-time.

9. The method of claim 6, wherein the support device further comprises a display, and wherein the method further comprises:
receiving patient data from a database on the network via the network interface, the patient device including at least data uploaded from a home monitoring device; and
displaying the patient data on the display of the support device.

10. The method of claim 9, further comprising transmitting the patient data to the remote location.

11. A system for remotely supporting a medical device, the system comprising:
a mobile cart including a tabletop, storage for one or more devices, and wheels, the cart supporting a display, a camera, a user interface, and a rechargeable battery; and
a remote support device including:
at least one video input configured to receive at least a video signal from a programmer for an implantable cardiac device;
a network interface; and
one or more hardware processors configured with instructions to transmit one or more video signals received at the at least one video input over a network to a remote location via the network interface.

12. The system of claim 11, wherein the storage for one or more devices comprises one or more shelves.

13. The system of claim 11, wherein the storage for the one or more devices comprises a cabinet, and wherein the at least one video input and the network interface are positioned on an I/O panel on an exterior surface of the cabinet.

14. The system of any of claim 11, wherein at least one of the one or more devices comprises a programmer for implantable cardiac devices.

15. The system of any of claim 11, wherein the remote support device includes at least one audio input, and wherein the one or more hardware processors are further configured to transmit one or more audio signals received at the at least one audio input over the network to the remote location via the network interface.
